(19) United States
(12) Patent Application Publication
(10) Pub. No.: US 2016/0031889 A1
(43) Pub. Date: Feb. 4, 2016

(54) ANTIVIRAL INDOLO[2,3-B]QUINOXALINE

(71) Applicant: VIRONOVA HERPES AB, Stockholm (SE)

(72) Inventors: Mohammed HOMMAN, NACKA (SE); Ngarita KINGI, ALVSJO (SE); Jan BERGMAN, SPANGA (SE); Robert BERG, RONNINGE (SE)

(73) Assignee: VIRONOVA HERPES AB, Stockholm (SE)

(21) Appl. No.: 14/775,310

(22) PCT Filed: Mar. 14, 2014
(86) PCT No.: PCT/EP2014/055178

§ 371 Date: Sep. 11, 2015

Related U.S. Application Data
(60) Provisional application No. 61/788,867, filed on Mar. 15, 2013.

(51) Int. Cl. C07D 487/04; A61K 45/06; A61K 31/4985

(52) U.S. Cl. CPC C07D 487/04; A61K 31/4985; A61K 45/06

(57) ABSTRACT

A compound of formula (I)

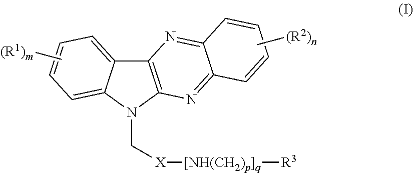

useful in the treatment of a herpes viral infection. A pharmaceutical composition including the compound of formula (I).

### TECHNICAL FIELD OF THE INVENTION

The present invention relates to indoloquinoxaline derivatives, to methods for preparing them as well as to their pharmaceutical use. In particular, the invention relates to novel indoloquinoxaline derivatives and their use in the treatment of viral infections.

### BACKGROUND OF THE INVENTION

As is well-known, viruses are the etiologic cause of many, sometimes life-threatening, diseases in both humans and animals. For example, herpes viruses such as herpes simplex 1 (HSV1), herpes simplex 2 (HSV-2), cytomegalovirus (CMV), Epstein-Barr virus (EBV), varicella zoster virus (VZV) and human herpes virus 6 (HHV 6) are associated with many common viral illnesses.

Following a primary infection with herpes simplex, the virus establishes latency in the sensory nerve cells for the rest of the patient's life and subsequently repeated virus reactivation can occur. Following a reactivation in the nerve cell the virus is transported through the nerves to the skin and then a lesion will develop. Immediately upon an outbreak of virus replication inflammation will follow. The inflammation contributes to the symptoms associated with herpes virus recurrence, including redness, swelling, itching and pain as well as lesions.

Herpes simplex viruses may be grouped into two serotypes, HSV type 1 (HSV-1) and type 2 (HSV-2), the clinical manifestations of which range from benign self-limiting orofacial and genital infections to potentially life threatening conditions such as encephalitis and generalized neonatal infections. Oral-facial HSV infections are primarily caused by HSV-1, which becomes latent after a primary infection e.g. in childhood. After reactivation a recurrent oral-facial HSV infection develops, more commonly known as a cold sore. About half of the patients experience early symptoms, e.g. pain, burning or itching at the site of the subsequent lesions. The condition is generally rapidly self-limiting and the healing time of a typical episode is about 10 days from the initial symptoms. Viral replication in the lip is initiated early and maximum virus load is attained 24 hours after the onset of the reactivation. The virus concentration is then dramatically reduced and typically virus cannot be isolated 70-80 hours after the onset.

The clinical presentation of genital HSV infections is similar to the oral-facial infections with some important exceptions. Genital HSV infections are most often caused by HSV-2 and following the primary infection the virus will latently infect sensory or autonomic ganglions. Reactivation will produce the local recurrent lesions on or near the genitals that are characteristic of the herpes infection.

A primary infection with varicella-zoster virus (VZV) causes chicken-pox. Like HSV, VZV becomes latent following the primary infection and can be activated as herpes zoster later on in life. Zoster usually results in skin rash and intensive acute pain. In 30% of the patients, the pain can be prolonged and continue for weeks or months after the rash has cleared up, or may even be permanent. HSV and VZV may, in addition to mucous or cutaneous manifestations, also cause keratitis in the eyes. This condition is also recurrent and may cause blindness.

There are a number of antiviral agents which are active against the human herpes viruses. However, so far clinical success in the treatment of recurrent herpes virus infections has been only limited and there still exists no cure for herpes. Various antivirals are used with varying success, e.g.: acyclovir (aciclovir), valacyclovir (valacyclovir), famciclovir, and penciclovir. For example, a cream formulation of acyclovir for topical application is sold by Ranbaxy under the generic name Zovirax.

PCT application WO 2005/123741 discloses alkyl substituted indoloquinoxalines of the general formula (I)

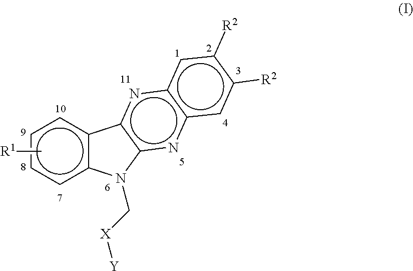

wherein R¹ is hydrogen or represents one or more similar or different substituents in the positions 7 to 10 selected from the group halogen, e.g. chloro, fluoro, bromo, lower alkyl/alkoxy, hydroxy, trifluoromethyl, trichloromethyl, trifluoromethoxy, R² represents similar or different C1-C4 alkyl substituents, X is CO or CH₂, Y is OH, NH₂, NH—(CH₂)ₙ—R³ wherein R³ represents lower alkyl, OH, NH₂, NHR⁴, or NR⁵R⁶ wherein R⁴, R⁵ and R⁶ independently are lower alkyl or cycloalkyl and n is an integer of from 2 to 4, with the provision that when X is CH₂, Y is OH or NH—(CH₂)ₙ—OH, and pharmacologically acceptable salts thereof. Said compounds are said to be useful for preventing and/or treating autoimmune diseases.

Some indoloquinoxalines also have been described for use as antiviral substances. Thus, in WO87/04436 the antiviral effect of a number of indoloquinoxalines, e.g. 2,3-dimethyl-6-(N,N-dimethylaminoethyl)-6H-indolo(2,3-b)quinoxaline (B-220), against herpes simplex virus of both type 1 and 2 is shown.

WO 2012/110631 discloses a pharmaceutical composition for topical administration comprising B-220 or a pharmaceutically acceptable salt thereof in a pharmaceutically acceptable carrier. The composition is useful for the treatment of herpes virus infections of the skin or mucous membranes in a mammal subject.

There however still remains a need for effective drugs and methods of treatment for primary as well as recurrent herpes infections.

### SUMMARY OF THE INVENTION

According to a first aspect, a compound is provided according to formula (I)

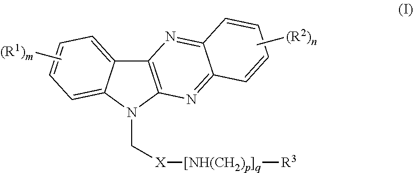

wherein
m is an integer of from 0 to 4;
n is an integer of from 0 to 4;
p is an integer of from 1 to 4;
q is 0 or 1;

### X is C═O, C═S or CH₂;

each R¹ is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;
each R² is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;

### R³ is OH, NH₂, NHR⁴, NR⁴R⁵, or (NR⁴R⁵R⁶)⁺Y⁻;

R⁴ is selected from C1-C6 alkyl;
R⁵ is selected from C1-C6 alkyl;
R⁶ is selected from C1-C6 alkyl, optionally substituted with a halogen or OH; C2-C6 alkenyl; and C2-C6 alkynyl;
Y is a pharmaceutically acceptable anion;
and pharmaceutically acceptable salts thereof;
with the proviso that if X is CH₂ and q is 0, R³ is OH or (NR³R⁴R⁵)⁺Y⁻,
for use in the treatment of a herpes viral infection.

In one particular embodiment, a compound of formula (I) is provided for topical administration to a mammal subject suffering from a primary or recurrent herpes virus infection, especially of oral-facial type.

According to a further aspect, the use of a compound as defined herein above is provided, for the manufacture of a medicament for use in the treatment of a herpes viral infection.

In one embodiment, if X is CH₂ and q is 0, then R³ is (NR³R⁴R⁵)⁺Y−.

In another embodiment, if if X is CH₂ and q is 0, then R³ is OH.

Some of the compounds of formula (I) are novel and therefore, in one aspect there is provided a compound of formula (I)

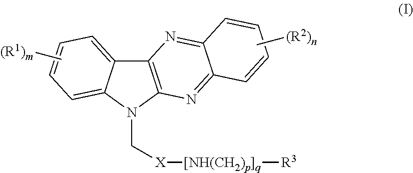

wherein
m is an integer of from 0 to 4;
n is an integer of from 0 to 4;
p is an integer of from 1 to 4;
q is 0 or 1;

### X is C═O, C═S or CH₂;

each R¹ is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;
each R² is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;

### R³ is OH, NH₂, NHR⁴, NR⁴R⁵, or (NR⁴R⁵R⁶)⁺Y⁻;

R⁴ is selected from C1-C6 alkyl;
R⁵ is selected from C1-C6 alkyl;
R⁶ is selected from C1-C6 alkyl, optionally substituted with a halogen or OH; C2-C6 alkenyl; and C2-C6 alkynyl;
Y is a pharmaceutically acceptable anion;
and pharmaceutically acceptable salts thereof;
with the proviso if R³ is different from (NR³R⁴R⁵)⁺Y⁻, X is C═S.

According to a still further aspect a pharmaceutical composition is provided comprising a compound according to formula (I) as defined herein above, with the proviso if R³ is different from (NR³R⁴R⁵)⁺Y⁻, X is C═S, in association with at least one pharmaceutically acceptable excipient.

In one embodiment, the pharmaceutical composition is an antiviral composition suitable for the treatment of a viral infection, e.g. a herpes viral infection.

In one particular embodiment, the composition of the invention is useful for topical administration to a mammal subject suffering from a primary or recurrent herpes virus infection, especially of oral-facial type.

In one embodiment, the pharmaceutical composition of the invention additionally comprises at least one additional therapeutically active ingredient suitable for topical administration.

According to one aspect, the invention relates to a compound of formula (I) as defined herein, or a pharmaceutically acceptable salt thereof, for use in the treatment of herpes virus infections of the skin or mucous membranes in a mammal subject by topical administration of a therapeutically effective dose thereof to the skin and/or mucous membrane of the mammal subject.

According to another aspect, the invention relates to a method of prophylactic and/or curative treatment of herpes virus infections of the skin or mucous membranes in a mammal subject comprising topical administration of a therapeutically effective dose of a compound of formula (I) as defined herein or a pharmaceutically acceptable salt thereof.

In one embodiment, the method also comprises topical administration, in combination or in sequence, of at least one additional pharmaceutically active ingredient suitable for topical administration, e.g. selected from antiviral agents, antibiotics, anaesthetic agents, analgesic agents, antiphlogistic agents, and anti-inflammatory agents.

Further aspects of the invention as well as embodiments thereof are described herein below and as defined in the claims.

### BRIEF DESCRIPTION OF THE DRAWING

FIG. 1 is a bar graph showing the viral titer as log₁₀ PFU/ml in HSV-1 infected in THCEC cells treated with inventive compounds, and with Acyclovir, respectively.

### DETAILED DESCRIPTION OF THE INVENTION

In the following the description, any reference to a compound of formula (I) also should be construed as a reference to a compound of any of the embodiments thereof, e.g. as represented by formulas (Ia)-(Is), unless otherwise indicated or apparent from the context.

Furthermore, unless otherwise indicated or apparent from the context, the following definitions shall apply throughout the specification and the appended claims.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

Unless otherwise stated or indicated, the term “C₁₋₆ alkyl” denotes a straight or branched alkyl group having from 1 to 6 carbon atoms. Examples of such C₁₋₆ alkyl according to the invention include methyl, ethyl, n-propyl, iso-propyl, n-butyl, iso-butyl, sec-butyl, t-butyl and straight- and branched-chain pentyl and hexyl.

By “alkyl substituted with at least one halogen” is meant an alkyl radical of the formula CₙXₚH₍₂ₙ₊₁₋ₚ₎—, wherein Xₚ refers to p independently selected halogen atoms, e.g. fluorine, replacing p hydrogen atoms of the alkyl radical CₙH₂ₙ₊₁— at the same or different carbon atoms. An example of an alkyl substituted with at least one halogen is trifluoromethyl. The alkyl substituted with at least one halogen may be a moiety forming a part of another radical, such as in trifluoromethoxy or difluoromethoxy.

The term “C2-C6 alkenyl” as used herein, alone or as part of another group, refers to a straight or branched chain hydrocarbon radical, containing 2, 3, 4, 5, or 6 carbons and at least one carbon to carbon double bond, e.g. vinyl, 2-propenyl, 3-butenyl, 2-butenyl, 4-pentenyl, 3-pentenyl, 2-hexenyl, 3-hexenyl, 2, and 2-buten-2-yl. All possible (E)- and (Z)-isomers is contemplated within the scope of the invention.

The term “C2-C6 alkynyl” as used herein refers to a straight or branched chain hydrocarbon radical, containing 2, 3, 4, 5, or 6 carbons, and at least one carbon to carbon triple bond, such as in 2-propynyl, 3-butynyl, 2-butynyl, 4-pentynyl, 3-pentynyl, 2-hexynyl, and 3-hexynyl, 3-methyl-1-butynyl, and 2-methyl-4-pentynyl, and the like. The alkynyl can in addition to carbon to carbon triple bonds also include a to carbon double bond.

The term “C3-C6 cycloalkyl” as employed herein alone or as part of another group refers to saturated cyclic hydrocarbyl groups, mono- or bicyclic rings(s), having a total of 3, 4, 5, or 6, carbons forming the ring, such as in cyclopropyl, cyclobutyl, cyclopentyl and cyclohexyl.

The terms alkoxy (or alkyloxy), alkenyloxy, alkynyloxy and cycloalkyloxy refer to a radical of the type RO—, wherein R is alkyl, alkenyl, alkynyl or cycloalkyl.

Unless otherwise stated or apparent from the context, the term “halogen” (or “halo”) means fluorine (fluoro), chlorine (chloro), bromine (bromo) or iodine (iodo).

In formula (I) each R¹ is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen.

In some embodiments, each R′ is independently selected from halogen and C1-C3 alkyl, C2-C3 alkenyl, C2-C3 alkynyl, C3-C4 cycloalkyl, C1-C3 alkoxy, C2-C3 alkenyloxy, C2-C3 alkynyloxy, C3-C4 cycloalkyloxy, and OH, any alkyl, alkenyl, alkynyl or cycloalkyl optionally being substituted by at least one halogen.

In some embodiments, each R¹ is independently selected from halogen, methyl, ethyl, methoxy, ethoxy, and OH, wherein any methyl, ethyl, methoxy and ethoxy optionally is substituted by at least one halogen. For example, each R¹ may be independently selected from halogen, methyl, methoxy, and OH, wherein any methyl and methoxy optionally is substituted by one or more halogen.

In some embodiments, each R¹ is as defined herein above, but is not OH, e.g. each R¹ is selected from halogen and C1-C6 alkyl, e.g. halogen and C1-C3 alkyl, in particular halogen and methyl. In some embodiments, each R¹ is halogen, e.g. F or Cl, in particular Cl.

The number of moieties R¹ in a compound of formula (I), indicated by the integer m, is from 0 to 4, e.g. from 0 to 3, or from 0 to 2, in particular m is 0 or 1.

In some embodiments, m is 1. In some other embodiments, m is 0, and the compound of formula (I) may then be represented by formula (Ia)

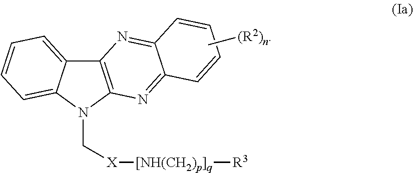

In some embodiments, the compound of formula (I) comprises one R¹ in position 9 on the 6H-indolo[2,3-b]quinoxaline ring, and optionally 1, 2 or 3 further R¹ at any of the other available positions (positions 7, 8 and 10). In some embodiments, m is 1 and R¹ is in position 9, and the compound of formula (I) may then be represented by formula (Ib)

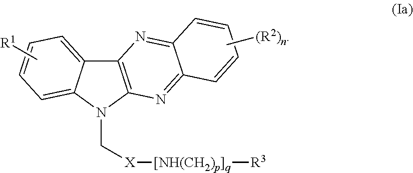

In a compound of formula (I), each moiety R² is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen.

For example, any R² may be selected from halogen, C1-C3 alkyl, C2-C3 alkenyl, C2-C3 alkynyl, C3-C5 cycloalkyl, C1-C3 alkoxy, C2-C3 alkenyloxy, C2-C3 alkynyloxy, C3-C5 cycloalkyloxy, and OH, any alkyl, alkenyl, alkynyl or cycloalkyl optionally being substituted by at least one halogen.

In some embodiments, any R² is selected from C1-C6 alkyl, C2-C6 alkenyl, C2-C6 alkynyl, C3-C6 cycloalkyl, or from C1-C3 alkyl, C2-C3 alkenyl, C2-C3 alkynyl, C3-C5 cycloalkyl, any alkyl, alkenyl, alkynyl or cycloalkyl optionally being substituted by at least one halogen.

In some embodiments, any R² is selected from C1-C6 alkyl, or from C1-C3 alkyl, said alkyl, optionally being substituted by at least one halogen; e.g. any R² is methyl.

The number of moieties R² in a compound of formula (I), indicated by the integer n, is from 0 to 4. For example, n is from 1 to 4, e.g. from 1 to 3, or from 2 to 3, e.g. n is 2. In some embodiments, n is at least 2 and the compound of formula (I) is substituted at least in positions 2 and 3 on the 6H-indolo[2,3-b]quinoxaline ring. In some embodiments when, n is 2, the compound of formula (I) is represented by formula (Ic)

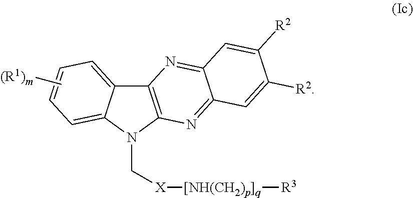

In some other particular embodiments, where m is 1, R¹ is in position 9, and n is 2 and R² is in positions 2 and 3, the compound of formula (I) may be represented by formula (Id)

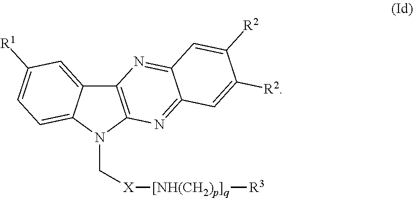

In still other embodiments, where m is 0, n is 2 and R² is in positions 2 and 3, the compound of formula (I) may be represented by formula (Ie)

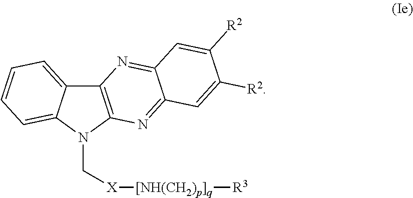

The compound of formula (I) comprises a moiety of formula (II):

—(CH₂)—X—[NH(CH₂)ₚ]_{q}—R³ (II)

attached in position 6 on the 6H-indolo[2,3-b]quinoxaline ring.

In the moiety of formula (II), X is C═O, C═S or CH₂. In some embodiments, X is CH₂, and the compound of formula (I) may then be represented by formula (If)

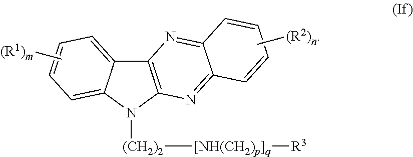

In some embodiments of a compound of formula (If), q is 0.

In some other embodiments of a compound of formula (I), X is C═O or C═S, and the compound may then be represented by formula (Ig)

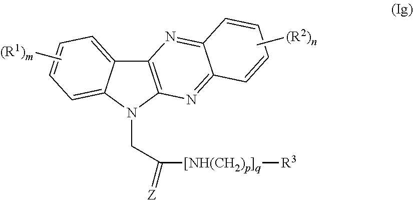

wherein Z is O or S.

In some embodiments of a compound of formula (Ig), Z is O.

In some other embodiments of a compound of formula (Ig), Z is S and the compound may then be represented by the formula (Ih)

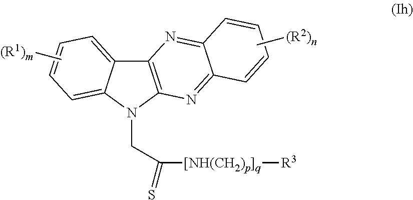

In some embodiments of a compound of formula (Ig) or (Ih), q is 0. In some other embodiments of a compound of formula (Ig) or (Ih), q is 1.

In some embodiments of a compound (Ig) or (Ih), m is 0 or 1, e.g. m is 1, e.g. m is 1 and R¹ is in position 9; and n is 2, e.g. n is 2 with one R² in position 2 and one R² in position 3.

In the moiety of formula (II), q is 0 or 1. When q is 0, the compound of formula (I) may be represented by formula (Ii)

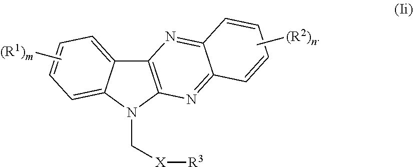

When q is 1, the compound of formula (I) may be represented by formula (Ij)

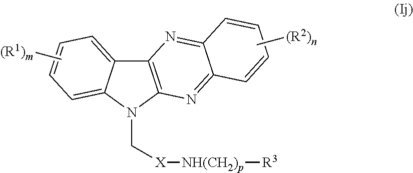

In the moiety of formula (II), p is an integer of from 1 to 4, or from 1 to 3. In some embodiments, p is an integer from 2 to 4, e.g. p is 2 or 3. In some embodiments, p is 2 and the compound of formula (Ij) may then be represented by formula (Ik)

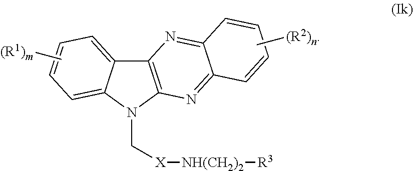

In the moiety of formula (II), R³ is OH, NH₂, NHR⁴, NR⁴R⁵, or (NR⁴R⁵R⁶)⁺Y⁻. In some embodiments, R³ is selected from NH₂, NHR⁴, NR⁴R⁵, and (NR⁴R⁵R⁶)⁺Y⁻, or from NHR⁴, NR⁴R⁵, and (NR⁴R⁵R⁶)⁺Y⁻. In some other embodiments, R³ is selected from NR⁴R⁵, and (NR⁴R⁵R⁶)⁺Y⁻. In still other embodiments, R³ is selected from NH₂, NHR⁴, and NR⁴R⁵.

In some embodiments of the compound of formula (I), R³ is NR⁴R⁵, and the compound may then be represented by formula (Im)

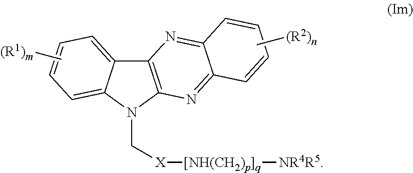

In some embodiments of the compound of formula (I), R³ is (NR⁴R⁵R⁶)⁺Y⁻, and the compound may then be represented by formula (In)

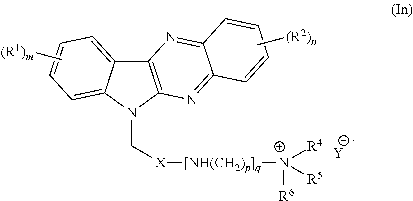

The ion Y⁻ in formula (I) may be any suitable pharmaceutically acceptable anion, such as Cl⁻, Br⁻, I⁻, methylsulfate, methanesulfonate, toluenesulfonate, acetate, or methylmethosulfate. In some embodiments Y⁻ is Cl⁻, Br⁻, or I⁻; e.g. Br⁻ or I⁻, in particular I⁻. In some embodiments, Y⁻ is methylsulfate.

In some embodiments of the invention, in the moiety of formula (II), X is CH₂, and R³ is (NR⁴R⁵R⁶)⁺Y⁻; the compound of formula (I) may then be represented by formula (Io)

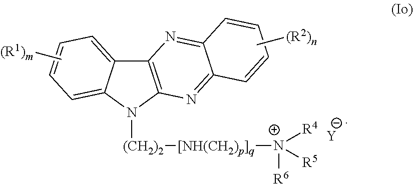

It should be noted that when, in the moiety of formula (II), X is CH₂ and q is 0, R³ is selected from OH and (NR⁴R⁵R⁶)⁺Y⁻, e.g. R³ is (NR⁴R⁵R⁶)⁺Y⁻.

In some embodiments of the invention, in the moiety of formula (II), X is CH₂, q is 0, and R³ is (NR⁴R⁵R⁶)⁺Y⁻, and the compound of formula (I) may then be represented by formula (Ip)

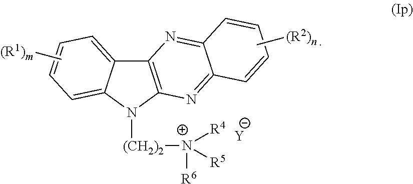

In a compound of formula (I) the moiety R⁴, when present, is selected from C1-C6 alkyl, or from C1-C5 alkyl, or from C1-C4 alkyl, or from C1-C3 alkyl, e.g. C1-C2 alkyl, e.g. R⁴ is methyl.

Likewise, the moiety R⁵, when present, is selected from C1-C6 alkyl, or from C1-C5 alkyl, or from C1-C4 alkyl, or from C1-C3 alkyl, e.g. C1-C2 alkyl, e.g. R⁵ is methyl.

Likewise, the moiety R⁶, when present, is selected from C1-C6 alkyl, optionally substituted with halogen or OH; C2-C6 alkenyl; and C2-C6 alkynyl.

In some embodiments, R⁶, when present, is selected from C1-C6 alkyl, optionally substituted with halogen or OH. In some embodiments, R⁶, when present, is selected from C1-C6 alkyl.

In some other embodiments, R⁶ is a moiety —(CH₂)—R⁷, wherein R⁷ is a C1-C5 alkyl substituted with halogen, such as Br, or with OH. In some embodiments, R⁷ is a C1-C5 alkyl substituted with halogen, such as Br. In some embodiments, R⁷ is a C1-C5 alkyl substituted with OH.

In still other embodiments, R⁶ is a moiety —(CH₂)_{w}—R⁸, wherein w is an integer of from 1 to 6, or from 2 to 4, or from 2 to 3; and R⁸ is halogen, such as Br, or OH. In some embodiments, R⁸ is halogen. In some other embodiments, R⁸ is OH.

When R⁶ is C2-C6 alkenyl or C2-C6 alkynyl, it more particularly may be C2-C3 alkenyl or C2-C3 alkynyl, e.g. vinyl (i.e. ethenyl), allyl (i.e.propenyl), ethynyl, or propynyl.

In some embodiments of a compound of formula (I), e.g in a compound of formula (Io) or (Ip), m is 0 or 1, e.g. m is 0; and n is 2, e.g. R² is in position 2 and 3.

In some other embodiments of the invention, in formula (I) m is 1 and R¹ is in position 9; n is 2 and R² is in position 2 and 3; X is C═Z wherein Z is 0 or S; and q is 1, i.e. the compound of formula (I) may be represented by formula (Iq)

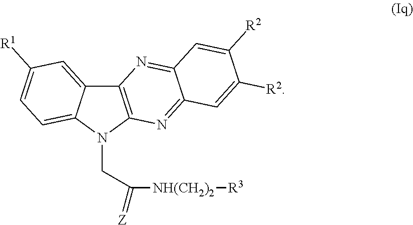

In still other embodiments of the invention, in formula (I) m is 0; n is 2 and R² is in position 2 and 3; X is CH₂; and q is 0, i.e. the compound of formula (I) may be represented by formula (Ir)

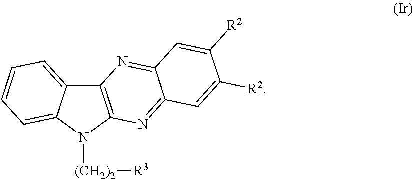

In some other particular embodiments of the invention, in formula (I) m is 0; n is 2 and R² is in position 2 and 3; Xis CH₂; q is 0, and R³ is (NR⁴R⁵R⁶)⁺Y⁻, i.e. the compound of formula (I) may be represented by formula (Is)

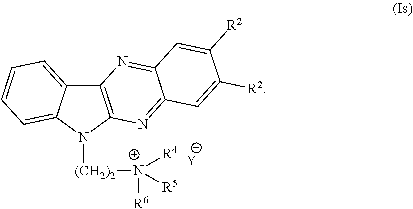

According to one aspect of the invention, novel compounds are provided. In these compounds X is different from C═S only when R³ is (NR⁴R⁵R⁶)⁺Y⁻. Thus, novel compounds of the invention are either compounds of formula (Ih) or compounds of formula (In), e.g. compounds of formula (Io) or (Ip).

Examples of novel compounds of the invention are
2,3-Dimethyl-6-[2-(trimethylamino)ethyl]-6H-indolo[2,3-b]quinoxaline iodide,
9-chloro-N-[2-(dimethylamino)ethyl]-2,3-dimethyl-6H-Indolo[2,3-b]quinoxaline-6-thioacetamide,
2,3-dimethyl-6-[2-(trimethylamino)ethyl]-6H-indolo[2,3-b]quinoxaline methyl sulfate, and
N-(2-(2,3-dimethyl-6H-indolo[2,3-b]quinoxalin-6-yl)ethyl)-N,N-dimethylprop-2-en-1-aminium bromide.

Examples of compounds of the invention for use as antiviral agents in the treatment of herpes infections are
2,3-Dimethyl-6-[2-(trimethylamino)ethyl]-6H-indolo[2,3-b]quinoxaline iodide,
9-chloro-N-[2-(dimethylamino)ethyl]-2,3-dimethyl-6H-Indolo[2,3-b]quinoxaline-6-acetamide,
9-chloro-N-[2-(dimethylamino)ethyl]-2,3-dimethyl-6H-Indolo[2,3-b]quinoxaline-6-thioacetamide,
2,3-dimethyl-6-[2-(trimethylamino)ethyl]-6H-indolo[2,3-b]quinoxaline methyl sulfate, and
N-(2-(2,3-dimethyl-6H-indolo[2,3-b]quinoxalin-6-yl)ethyl)-N,N-dimethylprop-2-en-1-aminium bromide.

Compounds of the present invention may be prepared by the person of ordinary skill in the art, e.g. by methods as described in PCT/SE87/00019 (WO87/04436), PCT/SE2005/000718 (WO 2005/123741), and PCT/EP2012/051864 (WO2012/104415) which documents are incorporated herein by reference. For example, compounds of the present invention wherein X is C═S may be prepared by reacting corresponding compounds wherein X is C═O, with a thionating agent, such as P₂S₅.2C₅H₅N, in a reaction such as described in PCT/EP2012/051864 (WO2012/104415).

Compounds of the present invention wherein R³ is (NR⁴R⁵R⁶)⁺Y⁻ may be prepared by reacting corresponding compounds wherein R³ is NR⁴R⁵, with a compound of formula R⁶Y, or by reacting corresponding compounds wherein R³ is NR⁵R⁶, with a compound of formula R⁴Y.

In one embodiment of the invention, the pharmaceutical composition is for the treatment of a virus selected from herpes viruses, such as herpes simplex 1 (HSV-1), herpes simplex 2 (HSV-2), cytomegalovirus (CMV), Epstein-Barr virus (EBV), varicella zoster virus (VZV) and human herpes virus 6 (HHV 6). In one embodiment of the invention, the virus is a herpes simplex 1 (HSV-1).

The compounds of the invention are useful as antiviral agents and thus, according to one aspect of the invention, an antiviral pharmaceutical composition is provided comprising a compound of formula (I) and at least one pharmaceutically acceptable excipient.

The pharmaceutically acceptable excipients may be for example, vehicles, adjuvants, carriers or diluents, such as are well-known to the person skilled in the art and as described e.g. in Remington: The Science and Practice of Pharmacy, 21th ed., Mack Printing Company, Easton, Pa. (2005). Further, it is contemplated that the pharmaceutical composition of the invention, in addition to a compound of formula (I), may contain also other therapeutically active substances, e.g. other antiviral agents.

The pharmaceutical composition of the invention may be administered parenterally or orally and may be used in a local or systemic antiviral treatment of a vertebrate in need of such treatment, e.g. a bird or a mammal, such as a human or an animal such as a domestic animal or a farm animal. It is contemplated that a pharmaceutical composition of the invention may be administered together with other, compatible drugs, such as another antiviral drug in multidrug therapy.

Pharmaceutically acceptable salts of the compound of the present invention may be formed using any organic or inorganic, pharmaceutically acceptable acid, such as are well-known to the person of ordinary skill in the art. Pharmaceutically acceptable acid addition salts according to the invention are salts that are safe and effective for topical use in mammals and that possess the desired biological activity, e.g. hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, or p-toluenesulfonate salts.

The pharmaceutical composition of the invention comprises a compound of formula (I) as defined herein and at least one pharmaceutically acceptable excipient. In one embodiment of the invention, the pharmaceutical composition comprises the compound of formula (I) or a pharmaceutically acceptable salt thereof in a pharmaceutical carrier suitable for topical delivery of the active ingredient.

In one embodiment, the pharmaceutical composition comprises the compound of formula (I) or a pharmaceutically acceptable salt thereof and an additional therapeutically active ingredient, suitable for topical administration, e.g. selected from antiviral agents, antibiotics, anaesthetic agents, analgesic agents, antiphlogistic agents and anti-inflammatory agents.

In one embodiment, the additional therapeutically active ingredient comprises or is an antiviral agent. The antiviral agents suitable for the purposes of the present invention are topically acceptable antiviral compounds, which in addition to being specific inhibitors of herpes virus multiplication also are active after topical administration and in addition are pharmaceutically acceptable for topical administration. This means that the toxicity of the antivirals must be sufficiently low to allow for a continuous contact with the human body and in particular with the skin and mucous membranes. Examples of antiviral agents are substances within the group comprising compounds acting on viral DNA polymerase, such as nucleoside analogues after phosphorylation to their triphosphate forms; phosphono formic and phosphonoacetic acids and their analogues; and other antiviral compounds having a different mechanism of action. As examples of antiviral agents which can be used in the combination of the invention can be mentioned acyclovir (ACV), ACV-phosphonate, brivudine (bromovinyldeoxyuridine, BVDU), carbocyclic BVDU, buciclovir, CDG (carbocyclic 2′-deoxyguanosine), cidofovir (HPMPC, GS504), cyclic HPMPC, desciclovir, edoxudine, famciclovir, ganciclovir (GCV), GCV-phosphonate, genivir (DIP-253), H2G (9-[4-hydroxy-2-(hydroxy-methyl)butyl]-guanine), HPMPA, lobucavir (bishydroxymethylcyclobutylguanine, BHCG), netivudine (zonavir, B W882C87), penciclovir, PMEA (9-(2-phosphonylmethoxy-ethyl)adenine), PMEDAP, sorivudine (brovavir, BV-araU), valacyclovir, 2242 (2-amino-7-(1,3-dihydroxy-2-propoxymethyl)purine), HOE 602, HOE 961; BPFA (batyl-PFA), PAA (phosphonoacetate), PFA (phosphonoformate); arildone, amantadine, BILD 1263, civamide (capsaicin), CRT, ISIS 2922, peptide T, tromantadine, virend, 1-docosanol (lidakol) and 348U87 (2-acetylpyridine-5-[2-chloro-anihno-thiocarbonyl]-thiocarbono-hydrazone).

Preferred antiviral agents are those with specific antiviral activity such as herpes specific nucleoside analogues which are preferentially phosphorylated in virus-infected cells and have very low or non-existent incorporation into cellular DNA as well as other compounds with specific antiviral activity. Acyclovir, for instance, has a selectivity ratio for the inhibitory activity against HSV-1 in vitro of about 2000. Among said preferred substances can in addition to acyclovir be mentioned brivudine, cidofovir, desciclovir, famciclovir, ganciclovir, HOE 961, lobucavir, netivudine, penciclovir, PMEA, sorivudine, valacyclovir, 2242, BPFA, PFA, PAA.

A suitable antiphlogistic agent, i.e. an agent capable of reducing inflammation, pain and/or fever, e.g. may be a non-steroidal anti-inflammatory drug (NSAID), such as diclofenac (IUPAC name 2-(2,6-dichloranilino)phenylacetic acid), or ibuprofen, (IUPAC name (RS)-2-(4-(2-methylpropyl)phenyl)propanoic acid), or a pharmaceutically acceptable salt thereof, e.g. a sodium, potassium or diethylamine salt thereof

A suitable anaesthetic agent e.g. may be lidocaine (IUPAC name 2-(diethylamino)-N-(2,6-dimethylphenyl)acetamide).

A suitable antiinflammatory agent e.g. may be adenosine (IUPAC name: (2R,3R,4S,5R)-2-(6-amino-9H-purin-9-yl)-5-(hydroxymethyl)oxolane-3,4-diol.

The antiinflammatory agent also may be selected from antiinflammatory glucocorticoids. A suitable glucocorticoid can be either naturally occurring or synthetic and can be selected from any of the Group I-ID glucocorticoids, according to a classification system for topical glucocorticoids used in the Nordic countries, corresponding to less potent, low or moderately potent glucocorticoids. Examples of glucocorticosteroids are alclometasone, amicinonide, beclomethasone, betamethasone, budesonide, ciclesonide, clobetasone, clocortolone, cloprednol, cortison, desonide, desoximethasone, dexamethasone, diflorosane, diflucortolone, difluprednate, fludrocortisone, fludroxycortid, flumethasone, flunisolide, fluocino lone acetonide, fluocinonide, fluocortin, fluocortolone, fluprednidene, fluticasone, halcinonide, halobetasol, halometasone, hydrocortisone, methylpredniso lone, mometasone, paramethasone, prednisolone, prednicarbate, prednisone, prednylidene, rofleponide, tipredane and triamcinolone and their esters, salts and solvates, that is hydrates, where applicable.

Some preferred glucocorticoids are hydrocortisone, alclometasone, desonide, fluprednidene, flumethasone, hydrocortisone butyrate, clobetasone, triamcinolone acetonide, betamethasone, budesonide, desoximethasone, diflorosane, fluocinolone, fluocortolone, fluticasone, methylpredniso lone aceponate, mometasone and rofleponide; in particular hydrocortisone, budesonide and fluticasone.

A suitable antibiotic e.g. may be selected from clindomycin, erythromycin, mupirocin, bacitracin, polymyxin and neomycin.

The carrier of the pharmaceutical composition should be stable and pharmaceutically acceptable and suitable for topical application. It should also enable incorporation of sufficient amounts of the compound of the present invention or of the pharmaceutically acceptable salt thereof, and optionally additional active ingredient(s). In addition to conventional ingredients in creams, lotions, gels or ointments, aerosolizable liquids, and foams, compositions based on phospholipids, including sphingolipids can be advantageous. In a cream or ointment formulation the carrier may be white petrolatum.

A liquid carrier may include water, alcohols or glycols or water-alcohol/glycol blends, in which effective amounts of the active ingredient(s) according to the invention can be dissolved or dispersed, optionally with the aid of non-toxic surfactants. Adjuvants such as fragrances and antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, creams etc., for application directly to the skin and/or mucous membrane of the user.

A pharmaceutical composition of the invention can be used for the prophylaxis and/or treatment of herpes virus infections in mammals including man. In one embodiment the composition is used for the treatment of primary or recurrent herpes virus infections. The treatment of infection should take place during the virus replication, preferably from the first appearance of redness/lesion or prodromal symptoms and for a period of 3-4 days at least. The formulation may be repeatedly applied, e.g. up to every two hours, during the whole episode until healing.

Prophylactic treatment may be performed in patients having regularly recurrent disease. In this case the formulation is applied to the area where a recurrence is expected before the appearance of the first symptoms. The compositions of the invention can be used to treat all types of herpes virus that replicate in the skin or the mucous membrane, e.g. HSV-1, HSV-2 and VZV.

The pharmaceutical compositions for topical administration according to the present invention are preferably creams, lotions, gels, sprays, foams, ointments or drops. The pharmaceutical compositions can be incorporated into plasters or patches to be applied to the skin of a patient to be treated for herpes infections or into pens or sticks for application to the skin or mucous membranes.

Liquid compositions can be applied from absorbent pads, used to impregnate bandages and other dressings, or sprayed onto the affected area using pump-type or aerosol sprayers.

Topical administration refers in this context to dermal or mucosal administration to the skin or mucous membrane.

The person of ordinary skill in the art will be well able to select suitable excipients in view of the selected formulation and form of administration, referring to e.g. handbooks such as Remington: The Science and Practice of Pharmacy 21st Edition. Philadelphia, Pa. Lippincott Williams & Wilkins. 2005.

In embodiments where a glucocorticoid is included in the pharmaceutical composition of the invention, care must be taken to define the optimal dose of the respective components, due to the herpes virus-stimulating effects of glucocorticoids. Too high a dose of the glucocorticoid might stimulate virus multiplication to an extent that can not be inhibited by the antiviral agent. With too low a dose the desired reduction of the symptoms of inflammation might not be achieved.

A pharmaceutical composition according to the present invention should contain a therapeutically effective amount of the compound of formula (I) as defined herein. For example, the relative amount of said compound in a pharmaceutical composition according to the present invention can be within the range of 0.1-10% (w/w), preferably 0.5-5% (w/w), e.g. about 1% (w/w).

In embodiments where an additional therapeutically active ingredient, such as any of the above-mentioned agents, is present in the composition, its concentration can be e.g. within the range of 0.005-5% (w/w), or within the range of 0.01-2% (w/w) or 0.25-1% (w/w).

In still another aspect, the present invention refers to a method of prophylactic and/or curative treatment of herpes virus infections of the skin or mucous membranes in a mammal subject, e.g. a human, comprising topical administration, in combination or in sequence, of a therapeutically effective dose of the compound of formula (I), or a pharmaceutically acceptable salt thereof, and at least one additional pharmaceutically active ingredient as mentioned herein above.

Herein below the invention is further illustrated by examples that should however not be construed as limiting the invention, the scope of which is defined by the claims.

### EXAMPLES

### Example 1

### 2,3-Dimethyl-6-[2-(trimethylamino)ethyl]-6H-indolo[2,3-b]quinoxaline iodide

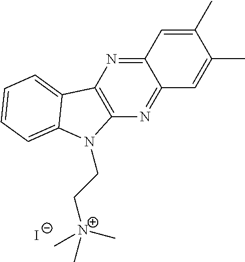 [SMILES: Cc1cc2nc3c4ccccc4n(CC[N+](C)(C)C)c3nc2cc1C ; [I-]]

Methyl iodide (0.32 ml, 5.2 mmol) was added to a solution of 2,3-dimethyl-6-(N,N-dimethylaminoethyl)-6H-indolo(2,3-b)quinoxaline (1.60 g, 5.0 mmol) in acetonitrile (50 ml). The solid thus formed was isolated by filtration.

Yield 1.83 g (80%); mp 281-283° C.; ¹H-NMR (DMSO-d₆) δ: 8.36 (d, 1H, J=7.52 Hz), 8.02 (s, 1H), 7.90-7.83 (m, 2H), 7.80 (t, 1H, J=7.22 Hz), 7.45 (t, 1H, J=7.46 Hz), 4.96 (t, 2H, J=6.94 Hz), 3.89 (t, 2H, J=6.99 Hz), 3.28 (s, 9H), 2.49 (s, 3H), 2.48 (s, 3H); ¹³C-NMR (DMSO-d₆) δ: 144.5 (s), 143.0 (s), 139.6 (s), 138.5 (s), 138.4 (s), 137.9 (s), 136.4 (s), 131.0 (d), 128.1 (d), 126.7 (d), 122.1 (d), 121.5 (d), 119.2 (s), 110.6 (d), 61.9 (t), 52.7 (q), 35.3 (t), 20.0 (q), 19.7 (q); IR νₘₐₓ: 3402, 3010, 1586, 1489, 1469, 1405, 1352, 923, 745 cm⁻¹.

### Example 2

### 9-chloro-N-[2-(dimethylamino)ethyl]-2,3-dimethyl-6H-Indolo[2,3-b]quinoxaline-6-acetamide

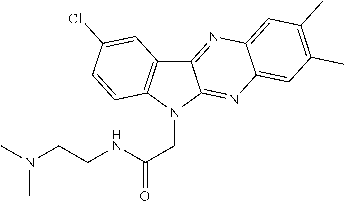

This compound was prepared as described in PCT/SE2005/000718 (WO 2005/123741), cf. “Compound E” at page 12 of said WO pamphlet.

### Example 3

### 9-chloro-N-[2-(dimethylamino)ethyl]-2,3-dimethyl-6H-Indolo[2,3-b]quinoxaline-6-thioacetamide

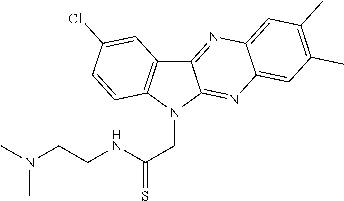

A diphosphorus pentasulfide dipyridinium complex (3 eq, 1.7 g, 4.5 mmol) was added to a solution of 9-chloro-N-[2-(dimethylamino)ethyl]-2,3-dimethyl-6H-indolo[2,3-b]quinoxaline-6-acetamide (0.61 g, 1.5 mmol) in dimethylsulfone (2.5 g) at 150° C. When the TLC analysis showed no starting material left the melt was cooled to room temperature (30 min). Water (50 mL) was added and the mixture was heated at reflux for 10 min. The solid thus formed was isolated by filtration and washed with water. The crude product was purified by chromatography using methanol and dichloromethane or methanol and dichloromethane as eluent (gradient from 1% to 10% methanol in dichloromethane).

Yield: 32% (0.20 g) (yellow solid); Mp: 182° C.; ¹H(DMSO-d₆) δ: 10.05 (1H, br s), 8.25 (1H, d), 7.94 (1H, s), 7.77 (1H, s), 7.68 (1H, dd), 7.52 (1H, d), 5.42 (2H, s), 3.69 (2H, t), 2.58 (2H, t), 2.45 (6H, s), 2.25 (6H, s); ¹³C-NMR (DMSO-d₆) δ: 196.2 (s), 145.2 (s), 142.5 (s), 139.8 (s), 138.8 (s), 137.9 (s), 137.6 (s), 136.4 (s), 130.2 (d), 128.0 (d), 126.6 (d), 125.4 (s), 120.9 (d), 120.4 (s), 112.3 (d), 55.7 (t), 50.9 (t), 44.8 (q), 42.8 (t), 20.0 (q), 19.7 (q). IR νₘₐₓ: 3217, 2945, 2821, 2770, 1585, 1534, 1458, 1207, 1167, 1119, 869, 796, 725, 684, 669 cm⁻¹.

### Example 4

### 2,3-dimethyl-6-[2-(trimethylamino)ethyl]-6H-indolo[2,3-b]quinoxaline methyl sulfate

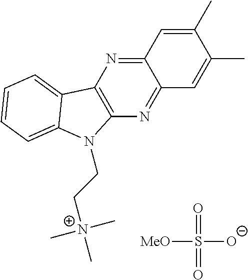 [SMILES: Cc1cc2nc3c4ccccc4n(CC[N+](C)(C)C)c3nc2cc1C ; COS(=O)(=O)[O-]]

2,3-dimethyl-6-(N,N-dimethylaminoethyl)-6H-indolo(2,3-b)quinoxaline (316 mg, lmmol) was dissolved in dioxane (20 ml) whereupon dimethyl sulfate (126 mg, 1 mmol) dissolved in dioxane (5 ml) was added dropwise at ambient temperature. The light-yellow precipitate formed was collected after 3 h.

Yield 313 mg (70%); Mp: 205-207° C.; ¹H-NMR (DMSO-d₆) δ: 8.40 (d, 1H, J=5.7 Hz), 8.04 (s, 1H), 7.88-7.85 (m, 2H), 7.80 (td, 1H, J=0.78, 7.22 Hz), 7.45 (t, 1H, J=5.5 Hz), 4.85 (t, 2H, J=4.47 Hz), 3.67 (t, 2H, J=4.45 Hz), 3.4 (s, 3H), 2.97 (s, 6H), 2.51 (s, 9H); ¹³C-NMR (DMSO-d₆) δ: 144.9 (s), 143.0 (s), 139.1 (s), 138.6 (s), 138.1 (s), 137.6 (s), 136.0 (s), 130.8 (d), 127.9 (d), 126.4 (d), 121.8 (d), 121.2 (d), 119.3 (s), 110.3 (d), 54.7 (t), 53.0 (q), 42.8 (q), 36.5 (t), 19.9 (q), 19.6 (q); IR νₘₐₓ: 3037, 2725, 1584, 1488, 1469, 1406, 1348, 1210, 999, 869, 748 cm⁻¹.

### Example 5

### N-(2-(2,3-dimethyl-6H-indolo[2,3-b]quinoxalin-6-yl)ethyl)-N,N-dimethylprop-2-en-1-aminium bromide

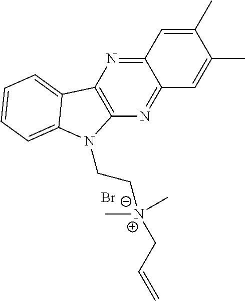

Yield: 56% (250 mg); Mp 248-252° C. ¹H-NMR (DMSO-d₆) δ: 8.39 (d, 1H, J=5.7 Hz), 8.07 (s, 1H), 7.92 (t, 2H, J=3.03 Hz), 7.82 (t, 1H, J=5.44 Hz), 7.46 (t, 1H, J=5.6 Hz), 6.16 (m, 1H, 6.16-6.17) 5.71 (m, 1H, 5.69-5.74) 4.99 (t, 2H, J=5.44 Hz), 4.23 (d, 2H, J=5.42 Hz), 3.81 (t, 2H, 5.47 Hz), 3.23 (s, 6H), 2.53 (d, 6H, 5.4 Hz); ¹³C-NMR (DMSO-d₆) δ: 144.7 (s), 143.2 (s), 139.3 (s), 138.7 (s), 138.5 (s), 138.1 (s), 136.2 (s), 130.9 (d), 128.2 (d), 128.1 (s), 126.8 (d), 125.7 (d), 121.9 (s), 121.4 (d), 119.5 (s), 110.7 (d), 66.2 (t), 60.2 (t), 50.5 (q), 35.6 (t), 19.8 (q), 19.5 (q); IR νₘₐₓ: 2998, 2958, 2912, 1613, 1585, 1487, 1469, 1407, 1375, 1326, 1250, 1207, 1147, 1121, 1005, 947, 757 cm⁻¹.

### Biological Tests

### Plaque Assay 1

The compounds prepared in Examples 1-4 were tested in a plaque assay using herpes simplex 1 virus (HSV1), and acyclovir (ACV) as a reference compound. When using the compounds of Example 2 and 3, no plaques could be seen. The plaque counting results obtained when using the compounds of Examples 1 and 4, and the reference compound ACV, respectively, are shown in Tables 1-3.

TABLE 1

| Ex. 1 | 10¹ dilution | 10² dilution | 10³ dilution |
|---|---|---|---|
| 1 uM | 19 | 3 | 1 |
| 5 uM | 8 | 2 | — |
| 25 uM | — | — | — |

TABLE 2

| Ex. 4 | 10¹ dilution | 10² dilution | 10³ dilution |
|---|---|---|---|
| 1 uM | 11 | — | — |
| 5 uM | 2 | — | — |
| 25 uM | — | — | — |

TABLE 3

| ACV | 10¹ dilution | 10² dilution | 10³ dilution |
|---|---|---|---|
| 1 uM | 15 | — | — |
| 5 uM | 8 | 1 | — |
| 25 uM | 2 | — | — |

### Plaque Assay 2

The compounds prepared in Examples 1-4 were tested in a further plaque assay, as follows:

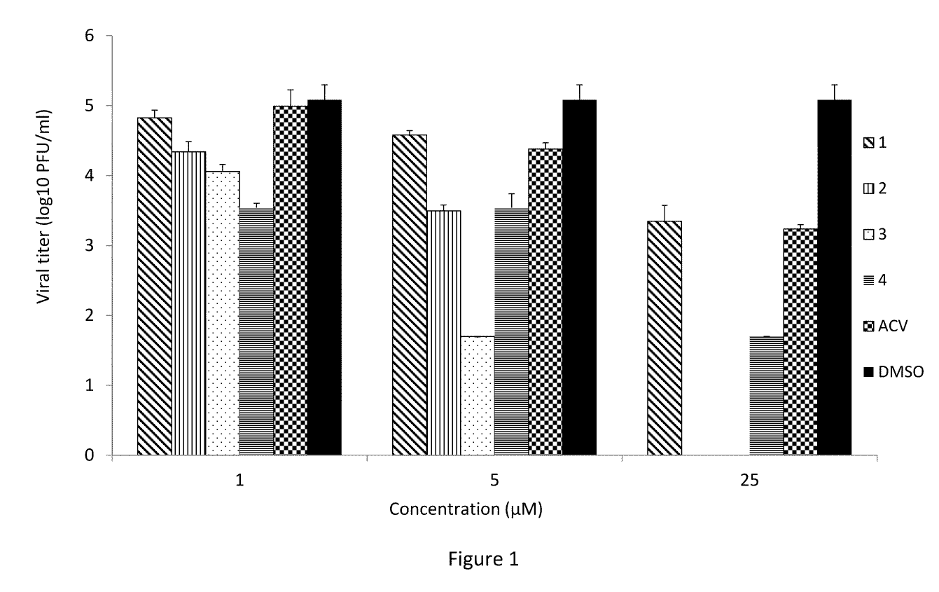

Prior to viral infection, 5×10⁴ cells/well of transduced human corneal endothelial (THCEC/GFP) cells were seeded in 24 well plates over night. Cell culture medium was removed and cells were infected with HSV-1 strain F at a multiplicity of infection (MOI) of 1 PFU/cell. One hour post-infection (hpi), cells were washed twice and fresh medium containing an increasing amount of compounds was added. All compounds were dissolved in cellculture graded DMSO. Cell culture medium from infected wells was collected at 24 hpi and stored at −80° C. until assessed for viral replication according to a standardized plaque assay for HSV-1. Briefly, virus dilutions were added onto 80% confluent Vero cells and incubated at 37° C. for 1 hour. After adsorption, the cells were overlain with DMEM medium containing 5% FBS and 1% agarose and incubated for three days, after which they were fixed with 10% formaldehyde, stained with 0.5% crystal violet, and the number of plaques were counted. Results are presented in FIG. 1 as log₁₀ PFU/ml.

1. A method of treating a herpes viral infection, comprising administering to a mammal in need thereof a therapeutically effective amount of a compound of formula (I) 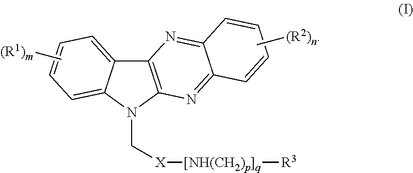
wherein
m is an integer of from 0 to 4;
n is an integer of from 0 to 4;
p is an integer of from 1 to 4;
q is 0 or 1;
X is C═O, C═S or CH₂;
each R¹ is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;
each R² is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;
R³ is OH, NH₂, NHR⁴, NR⁴R⁵, or (NR⁴R⁵R⁶)⁺Y⁻;
R⁴ is selected from C1-C6 alkyl;
R⁵ is selected from C1-C6 alkyl;
R⁶ is selected from C1-C6 alkyl, optionally substituted with a halogen or OH; C2-C6 alkenyl; and C2-C6 alkynyl;
Y is a pharmaceutically acceptable anion;
or a pharmaceutically acceptable salt thereof;
with the proviso that if X is CH₂ and q is 0, R³ is OH or (NR³R⁴R⁵)⁺Y⁻.

2. The method according to claim 1, wherein, X is CH₂.

3. The method according to claim 1, wherein X is C═O or C═S.

4. The method according to claim 1, wherein q is 0.

5. The method according to claim 1, wherein q is 1.

6. The method according to claim 1, wherein m is 0 or 1.

7. The method according to claim 1, wherein n is 2.

8. The method according to claim 1, wherein R³ is NH₂, NHR⁴, NR⁴R⁵, or (NR⁴R⁵R⁶)⁺Y⁻.

9. The method according to claim 1, wherein the compound is selected from
2,3-Dimethyl-6-[2-(trimethylamino)ethyl]-6H-indolo[2,3-b]quinoxaline iodide,
9-chloro-N-[2-(dimethylamino)ethyl]-2,3-dimethyl-6H-Indolo[2,3-b]quinoxaline-6-acetamide,
9-chloro-N-[2-(dimethylamino)ethyl]-2,3-dimethyl-6H-Indolo[2,3-b]quinoxaline-6-thioacetamide,
2,3-dimethyl-6-[2-(trimethylamino)ethyl]-6H-indolo[2,3-b]quinoxaline methyl sulfate, and
N-(2-(2,3-dimethyl-6H-indolo[2,3-b]quinoxalin-6-yl)ethyl)-N,N-dimethylprop-2-en-1-aminium bromide,
or a pharmaceutically acceptable salt thereof.

10. The method according to claim 1, wherein the herpes virus is herpes simplex 1.

11. The method according to claim 1, wherein the compound is administered together with at least one further therapeutically active agent, in sequence or in combination.

12. The method according to claim 11, wherein the at least one further therapeutically active agent is selected from antiviral agents, antibiotics, analgesics, anaesthetic agents, antiphlogistic agents and antiinflammatory agents.

13. The method according to claim 12, wherein the at least one further therapeutically active agent is an antiphlogistic agent.

14. The method according to claim 12, wherein the at least one further therapeutically active agent is an antiinflammatory agent.

15. The method according to claim 1, wherein the compound is administered by topical administration.

16. The method according to claim 15, wherein the treatment is dermal or mucosal treatment.

17. The method according to claim 15, wherein the compound is in a formulation in the form of a cream, liquid, lotion, gel, spray, foam or ointment.

18. The method according to claim 15, wherein administration is by use of a patch, stick, spray dispenser, tube, or pen containing the compound and at least one pharmaceutically acceptable excipient.

19. A compound of formula (I) 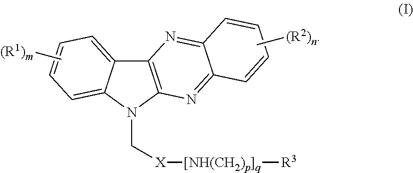
wherein
m is an integer of from 0 to 4;
n is an integer of from 0 to 4;
p is an integer of from 1 to 4;
q is 0 or 1;
X is C═O, C═S or CH₂;
each R¹ is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;
each R² is independently selected from halogen, C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C3-C6 cycloalkyloxy, and OH, any alkyl, alkenyl, alkynyl or cycloalkyl optionally being substituted by at least one halogen;
R³ is OH, NH₂, NHR⁴, NR⁴R⁵, or (NR⁴R⁵R⁶)⁺Y⁻;
R⁴ is selected from C1-C6 alkyl;
R⁵ is selected from C1-C6 alkyl;
R⁶ is selected from C1-C6 alkyl, optionally substituted with a halogen or OH; C2-C6 alkenyl; and C2-C6 alkynyl;
Y is a pharmaceutically acceptable anion;
or a pharmaceutically acceptable salt thereof;
with the proviso if R³ is different from (NR³R⁴R⁵)⁺Y⁻, X is C═S.

20. The compound according to claim 19, wherein X is CH₂.

21. The compound according to claim 19, wherein X is C═O or C═S.

22. The compound according to claim 19, wherein q is 0.

23. The compound according to claim 19, wherein q is 1.

24. The compound according to claim 19, wherein m is 0 or 1.

25. The compound according to claim 19, wherein n is 2.

26. The compound according to claim 19, wherein R³ is NH₂, NHR⁴, NR⁴R⁵, or (NR⁴R⁵R⁶)⁻Y⁻.

27. A compound according to claim 19, selected from
2,3-Dimethyl-6-[2-(trimethylamino)ethyl]-6H-indolo[2,3-b]quinoxaline iodide,
9-chloro-N-[2-(dimethylamino)ethyl]-2,3-dimethyl-6H-Indolo[2,3-b]quinoxaline-6-thioacetamide,
2,3-dimethyl-6-[2-(trimethylamino)ethyl]-6H-indolo[2,3-b]quinoxaline methyl sulfate, and
N-(2-(2,3-dimethyl-6H-indolo[2,3-b]quinoxalin-6-yl)ethyl)-N,N-dimethylprop-2-en-1-aminium bromide,
or a pharmaceutically acceptable salt thereof.

28. A compound according to claim 19, for use in therapy.

29. A compound according to claim 19, for use as an antiviral agent.

30. A pharmaceutical composition comprising a compound according to claim 19 and at least one pharmaceutically acceptable excipient.

31. The pharmaceutical composition according to claim 30, for topical administration.

32. The pharmaceutical composition according to claim 30, wherein the compound according to any one of the compounds 19 to 27 is present in an amount of 0.1-10% (w/w).

33. The pharmaceutical composition according to claim 30, comprising at least one additional therapeutically active ingredient.

34. The pharmaceutical composition according to claim 33, wherein the additional therapeutically active ingredient is selected from antiviral agents, antibiotics, analgesics, anaesthetic agents, antiphlogistic agents and antiinflammatory agents.

35. The pharmaceutical composition according to claim 34, wherein the additional therapeutically active ingredient is an antiphlogistic agent.

36. The pharmaceutical composition according to claim 34, wherein the additional therapeutically active ingredient is an antiinflammatory agent.

37. The pharmaceutical composition according to claim 33, wherein the additional therapeutically active agent is present in an amount of 0.005-5% (w/w).

38. The pharmaceutical composition according to claim 30, for use in the prophylaxis and/or treatment of a herpes virus infection in a mammal subject.

39. The pharmaceutical composition according to claim 30, for dermal or mucosal treatment.

40. The pharmaceutical composition according to claim 30, in the form of a cream, liquid, lotion, gel, spray, foam or ointment.

41. A patch, stick, spray dispenser, tube, or pen containing a pharmaceutical composition according to claim 30.

42. A method of treatment of a herpes virus infection by administration of a therapeutically effective amount of a compound according to claim 19, to a mammal in need of such treatment.
